(19) United States
(12) Patent Application Publication
(10) Pub. No.: US 2002/0098508 A1
(43) Pub. Date: Jul. 25, 2002

(54) Genome reading device

(76) Inventors: B. Nash Williams, Salt Lake City, UT (US); Michael D. McCully, Houston, TX (US)

Correspondence Address: Michael D. McCully, Suite 1023, 201 So. Heights Blvd., Houston, TX, 77007-5855, US

(21) Appl. No.: 10/057,857

(22) Filed: Jan. 23, 2002

Related U.S. Application Data
(60) Provisional application No. 60/263,677, filed on Jan. 24, 2001.

(51) Int. Cl. C12Q 1/68; G01N 33/48; G06F 19/00

(52) U.S. Cl. 435/6; 435/287.2; 702/20

(57) ABSTRACT

A self-contained device which can be utilized by the general population to analyze genetic materials or samples. The device incorporates apparatus for removing a biological sample, apparatus for analyzing the genetic make-up of the biological sample, computer and database for correlating the genetic makeup analysis with a specific physical condition or malady, and a device for reporting the specific condition information. The device can also incorporate automatic apparatus to remove and store biologically hazardous materials which may be created by handling of the genetic material and device components contaminated by the materials.

### CROSS-REFERENCE TO RELATED APPLICATIONS

This application claims the benefit of 35 U.S.C. §111(b) provisional application Serial No. 60/263,677, filed Jan. 24, 2001, entitled “Genome Reading Device”, incorporated herein by reference.

### STATEMENT REGARDING FEDERALLY-SPONSORED RESEARCH DEVELOPMENT

Not applicable.

### BACKGROUND OF THE INVENTION

### FIELD OF THE INVENTION

Several biological projects across the country are working to decode the human genetic codes. This effort has been referred to as the human genome.

Decoding is intended to determine what combinations of genetic material make up a human body. The understanding of the genome is believed to be the realization of a clearer understanding of human traits, features, and the cause of many genetically-originating diseases.

Genetic screening is currently utilized in limited applications to determine a patient's genetic predisposition to various diseases. Upon completion of the genome identification, the efforts of these biological projects are expected to focus on isolating the gene or combination of genes which may cause many maladies or may predispose an individual to such maladies, with the intention of creating genetically engineered cures for same.

Human genome analysis is constantly being refined, and is typically performed by depositing a body fluid or hair sample in a small container or plate for analysis. The analysis would provide the genome of an individual. This genome could then provide an individual with health information and other information regarding his predisposition for various diseases. This information is very personal in nature, and is currently available only at medical facilities, due to the fact that implementation and utilization of these human genome analysis technologies are very costly.

### SUMMARY OF THE INVENTION

The present invention comprises generally a self-contained device which can be utilized by the general population to analyze genetic materials or samples. The device incorporates apparatus for removing and/or accepting a biological sample, apparatus for analyzing the genetic make-up of the biological sample, computer and database for correlating the genetic make-up analysis with a specific physical condition or malady, and a device for reporting the specific condition information. The device can also incorporate automatic apparatus to remove and store biologically hazardous materials which may be created by handling of the genetic material and device components contaminated by the materials.

### BRIEF DESCRIPTION OF THE DRAWINGS

The single FIGURE is a schematic drawing of the genome reading device of the present invention.

### DETAILED DESCRIPTION OF THE INVENTION

The present invention provides a means to utilize human genome decoding technology in such a way as to make it readily available and economical for more wide-spread use in doctors' offices and clinics, and eventually to/for the general population. In one embodiment, the invention is provided in a generally contained fixture similar to an ATM, a kiosk, or the like, and includes an apparatus to receive or remove a biological sample from a person and deposit the sample on a device or plate for decoding the genetic information present in the sample. The genetic sample would be analyzed and the analysis compared to a computerized database that correlates specific genes or gene combinations with specific conditions or tendencies toward specific physical conditions or diseases. Analysis of the genetic sample (i.e., the specific conditions or disease-tendency information) would be provided to the person by use of a visual electronic device such as a visual monitor, and/or in printed text, and/or sent electronically to a recipient, such as a physician or insurer.

The apparatus to receive or remove a biological sample from a person embodies a means to assure sanitary reuse of the device, and embodies means for safe removal and disposal of all biologically hazardous materials from the device.

The present device may be utilized at small clinics, medical facilities, hospitals, or simply at convenient locations. The genetic information may be provided with general information concerning the screening results. The information would identify propensities toward specific emotional or physical traits, the genetic probability to acquire diseases, and if there is a high probability for specific disease, in order to provide further information about the disease, preventative measures and cures for the diseases, and facilities which specialize in treatments. In the event the genome analysis provides a negative result, the person receiving the information could be directed to the location of the nearest facility to corroborate the results or further verify those results by additional examination.

When used personally or individually, as in a kiosk, payment for use could be made to an attendant or by utilizing a credit card charging system or an electronic charging means such as to an insurer, doctor, or hospital.

The present device allows an individual to explore his individual genome in private without disclosure to others, such as insurers or doctors. The results can be accumulated through networking the devices, thereby providing further knowledge of the condition of various populations. The results can be made to exclude personal information about the those being tested to assure privacy.

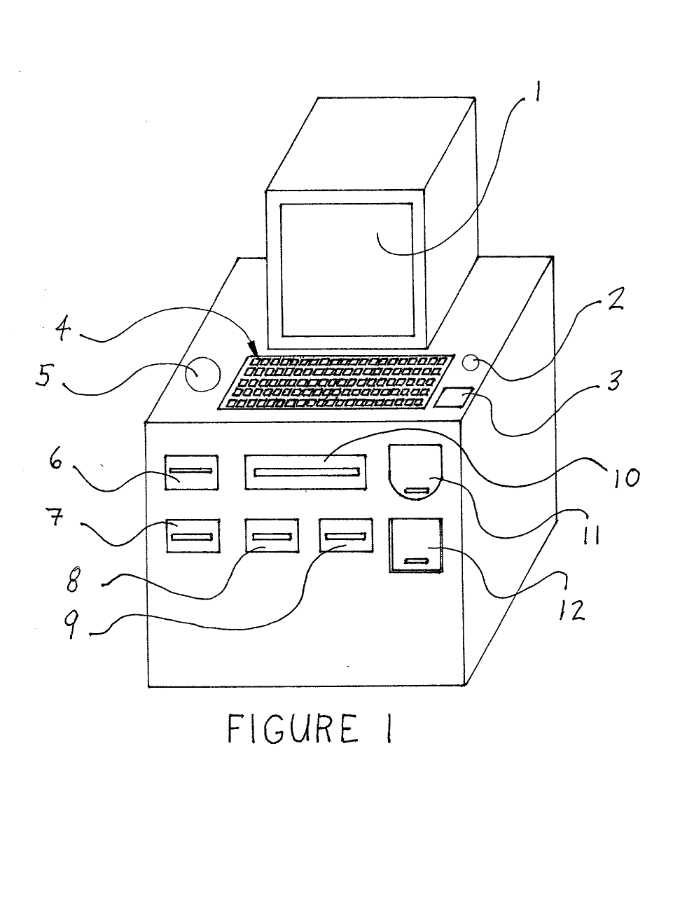

In FIG. 1, Item 1 is a visual electronic screen (monitor) for displaying information and for use as touch screen. Item 2 is a microphone which may be used with voice recognition systems for verbal commands. Item 3 is a pointing device to be used with the monitor. Item 4 is a keyboard. Item 5 is a speaker used for digital verbal directions and information. Item 6 is a slot used for credit card and electronic charges. Item 7 is a 3.5″ floppy read/write drive for input or output of information. Item 8 is a CD read/write drive for input or output of information. Item 9 is a Zip read/write drive for input or output of information. Item 10 is a printer/paper output device. Item 11 is a specimen removal apparatus. Item 12 is a specimen deposit apparatus. The database is internal or may be provided externally via an electronic connection (not shown).

In its simplest form, the genome reading device of the present invention is totally self-contained within a typical rectangular housing. The housing includes a biological sample reading device 12, which can be a conventional device for analyzing body fluids (e.g., blood, saliva) or other small biological samples (e.g., hair, tissue). The biological sample reading device can comprise multiple input devices, including the specimen removal apparatus 11 that punctures or pricks the end of a user's finger and draws a sample of blood. Such a device can comprise a tubular member that is drawn down around the finger to position the finger, followed by a sterile pin-prick to prick the end of the finger and draw blood onto a specimen tray or slide. The tubular member can also be drawn down further around the finger for a specified time-period in order to assist in forcing a sufficient quantity of blood from the finger onto the specimen tray.

The user communicates with the genome reading device by the use of various input devices—a keyboard 4, visual monitor 1, track-ball cursor pointing device 3, touch-screen monitor, voice-recognition system, and audio instructions from the genome reading device, for example.

Following the specimen deposit, the user withdraws his finger, and the blood sample is analyzed. During the sample analysis process, the sample input device prepares to reconfigure itself with a new sterile tubular member, sterile prick-pin, and sterile blood specimen tray, upon re-initiation of the operating sequence. The entire operating sequence can be partially or fully automated, with or without user input. Such mechanical automation is within the purview of the skilled robotics designer.

The blood sample (or other biological sample—saliva, hair, tissue, etc.) is analyzed in the conventional manner to determine the person's unique genetic DNA sequence. This unique genetic DNA sequence is then compared, using the device's self-contained computer processor, with the database of the approximately 3.5 billion chemical letters (A, C, G, T) that make up human DNA, to result in a unique genetic profile for the person. The resulting genetic profile can then be correlated to gene-specific or gene combination-specific physical conditions to indicate the person's propensity to any of a number of genetically-disposed conditions or diseases. Knowing these propensities, scientists and doctors can design treatment courses to prevent the particular indicated disease(s).

The DNA database is ever-expanding, having new genetic/disease information added to it frequently, and therefore, will likely be managed by a DNA database server, accessible via the Internet, as opposed to a self-contained database within the genome reading device. In such a configuration, the gene-searching and physical condition/disease correlating will likely be performed at the DNA database server, and the results automatically sent back to the genome reading device.

Following each biological sample analysis, the biological sample, specimen tray, and tubular finger member and prick-pin, if used, are all automatically discarded and collected in a secure medical waste (biological hazard) receptacle inside the housing.

The genome reading device of the present invention includes a plurality of output devices for outputting the resulting genetic profile and disease-propensity information. For example, the information could be displayed on the visual monitor and printed out on hard-copy. In addition, the analysis and resulting information could be digitally recorded onto computer diskettes or CD's, and/or electronically sent to specific locations—doctors' offices, hospitals, insurance companies, etc., or wherever the user chooses. Such electronic transformation of the analyses and resulting information into visual, hard-copy, and/or transmittable electronic media is commonly known.

Following recording/transmission of the analyses and resulting information, the genome reading device would automatically (or at user-option) delete/destroy all electronic data relating to the genetic sample and genome analysis, in order to insure the user's privacy.

Each free-standing genome reading device is electronically connected to a central information “clearing house” that keeps a record of how many times the unit has been used, and therefore, when the unit requires replenishing with sterile supplies and when the medical waste requires removal and disposal. The clearing house then signals a maintenance operator to replenish the genome reading device with sterile supplies and remove and dispose of the accumulated medical waste.

Such a genome reading device is suitable for use in a health-care professional's office or clinic, hospital, etc. It is contemplated, however, that the present invention have much broader use and application. A further embodiment is intended for use by the general public, and can be located as a stand-alone unit in a pharmacy, for instance, or a kiosk located in a shopping mall or waiting room of a hospital, for instance. In these more “commercial” settings, this further embodiment incorporates a mechanism for using a credit card for purchasing the services provided by the genome reading device. Such credit card using devices are in common use today, as for use with pay telephones, vending machines, ATM's, etc., making the genome reading device of the present invention a truly stand-alone and independent device.

The device can also be fully automated to (1) remove a genetic sample from the user; (2) analyze the human genome; (3) scan the genome analysis results and supplement the analysis results with general information concerning certain aspects of the analysis results; (4) upon direction from the user, (i) display certain genome analysis results and general information on a visual monitor, (ii) print the analysis results and general information on hard-copy, (iii) transfer the analysis results and general information to a portable machine-readable medium, (iv) electronically transmit the analysis results to specific designation(s), and (v) destroy all electronic data relating to the genetic sample and genome analysis; (5) automatically dispose of all biological and medical waste generated during the genetic sample removal and analysis; (6) self-clean and sterilize the device for subsequent automatic use by another user; and (7) process payment for use of the device.

What is claimed is:

1. A self-contained apparatus for analyzing genetic materials and providing genetic information, comprising:
means for analyzing the DNA sequences of a biological sample;
means for identifying specific genes within the DNA sequence;
means for correlating a specific gene with a specific physical condition;
means for reporting information regarding the specific condition.
means for removing, collecting, and storing biologically hazardous materials which may result from obtaining the genetic material, and for removing, collecting, and storing apparatus components potentially contaminated by the genetic material.

2. A self-contained apparatus for analyzing genetic materials and providing genetic information as set forth in claim 1, further comprising means for charging a credit card.

3. A self-contained apparatus for analyzing genetic materials and providing genetic information as set forth in claim 1, further comprising means for electronically transmitting the genetic information.

4. A self-contained apparatus for analyzing genetic materials and providing genetic information as set forth in claim 1, wherein the apparatus operates as a stand alone kiosk.

5. A self-contained apparatus for analyzing genetic materials and providing genetic information as set forth in claim 1, further comprising means for accumulating the genetic information.

6. A self-contained apparatus for analyzing genetic materials and providing genetic information as set forth in claim 1, further comprising means for printing the genetic information.

7. A self-contained apparatus for analyzing genetic materials and providing genetic information as set forth in claim 1, further comprising means for displaying the genetic information on a visual electronic screen.

8. A self-contained apparatus for analyzing genetic materials and providing genetic information as set forth in claim 1, further comprising a tubular component within which to insert a host's finger for removal of genetic material.

9. A self-contained apparatus for analyzing genetic materials and providing genetic information as set forth in claim 8, further comprising a small diameter pin to penetrate the finger for removal of genetic material.

10. A self-contained apparatus for analyzing genetic materials and providing genetic information as set forth in claim 8, further comprising means for reducing the diameter of the tubular component such for compressing the finger, thereby assisting removal of the genetic material.

11. A self-contained apparatus for analyzing genetic materials and providing genetic information as set forth in claim 1, further comprising means for cutting the genetic material from a host.

12. A method of genetic profiling, comprising:
a) obtaining a genetic sample from a user;
b) analyzing the sample genome to identify specific genes;
c) correlating specific genes with specific physical conditions;
d) providing information specific to the specific conditions;
e) providing the specific condition information in human-readable and/or machine-readable form;
f) removing, collecting, and storing biologically hazardous materials which may have resulted from obtaining the genetic sample, and removing, collecting, and storing apparatus components potentially contaminated by the genetic sample.

13. A method of genetic profiling as set forth in claim 12, further comprising processing payment for the services of gene profiling.

14. A method of genetic profiling as set forth in claim 12, wherein step f) is automatic following completion of steps a)-e).

15. A method of genetic profiling as set forth in claim 12, wherein step a) comprises withdrawing blood from the finger of the user.

16. A method of genetic profiling as set forth in claim 12, further comprising destroying all electronic data relating to the genetic sample and genome analysis.
